(19) United States
(12) Patent Application Publication
(10) Pub. No.: US 2019/0057762 A1
(43) Pub. Date: Feb. 21, 2019

(54) INFORMATION PROCESSING DEVICE

(71) Applicants: Toyosaki Accounting Office Co., Ltd., Tokyo (JP); GENERAL INCORPORATED ASSOCIATION LS GENERAL RESEARCH LABORATORY, Tokyo (JP)

(72) Inventor: Osamu TOYOSAKI, Tokyo (JP)

(73) Assignees: Toyosaki Accounting Office Co., Ltd., Tokyo (JP); GENERAL INCORPORATED ASSOCIATION LS GENERAL RESEARCH LABORATORY, Tokyo (JP)

(21) Appl. No.: 16/078,915

(22) PCT Filed: Feb. 21, 2017
(86) PCT No.: PCT/JP2017/006437

§ 371 Date: Aug. 22, 2018

(30) Foreign Application Priority Data
Feb. 26, 2016 (JP) 2016-035925

(51) Int. Cl. G16H 20/10; G16H 10/60; G16H 50/20

(52) U.S. Cl. CPC G16H 20/10; G16H 50/20; G16H 10/60

(57) ABSTRACT

The purpose of the present invention is to establish methods for: building medical big data taking privacy into consideration; deriving a more appropriate medicine dosage corresponding to medicine dosage and attribute information of patients; and discovering, in addition to the effect of the medicine on one disease symptom, the effect on another disease symptom. In the present invention, a data collection unit 40 collects health examination data and the like. A patient attribute information acquisition unit 61 acquires at least one attribute of a patient input from a patient terminal 1. A corresponding information acquisition unit 62 acquires, from a corresponding information database 82, corresponding information indicating a correspondence relationship between a medicine dosage having an effect on a disease symptom of which the patient is aware and the at least one attribute. An optimal dosage calculation unit 63 calculates the optimal medicine dosage in respect to the disease symptom of which the patient is aware, on the basis of the patient attribute information and the corresponding information. A separate effect analysis unit 72 analyzes, on the basis of information other than the patient attribute information, an effect separate from an effect analyzed by an effect analysis unit 44.

### TECHNICAL FIELD

The present invention relates to an information processing device.

### BACKGROUND ART

Heretofore, there have been medicine dosage-setting support devices that simply and precisely set doses of medical products to be administered to patients in accordance with disease symptoms, ages and the like of the patients (for example, see Patent Document 1).
Patent Document 1: Japanese Unexamined Patent Application, Publication No. 2004-267514

### DISCLOSURE OF THE INVENTION

### Problems to be Solved by the Invention

However, in regard to relationships between medicine dosages and attribute information of patients, because the attribute information entered for a patient is fixed, correspondence relationships between medicine dosages and attribute information of patients are not understood. Moreover, because a medicine does not affect only one disease symptom, there is always interest in discovering effects on other disease symptoms. Accordingly, there are calls for new technologies to address situations in which a more appropriate medicine dosage according to a medicine dosage and attribute information of patients should be derived, situations in which both the effect of a medicine on one disease symptom and an effect of the medicine on another disease symptom should be discovered, and so forth.

The present invention has been made in consideration of this situation, and an object of the invention is to establish methods for deriving a more appropriate medicine dosage corresponding to a medicine dosage and attribute information of patients and for discovering, in addition to the effect of a medicine on one disease symptom, an effect on another disease symptom.

### Means for Solving the Problems

In order to achieve the object described above, an aspect of an information processing device of the present invention includes data collection means that collects at least one of health examination data and medical consultation data relating to an individual in association with a second identifier that is capable of specifying the individual, the second identifier being generated on the basis of a first identifier that is assigned in order to specify the individual within a predetermined group.

In order to achieve the object described above, an aspect of the information processing device of the present invention includes: an information processing device for suggesting a treatment guideline for an individual on the basis of at least one of health examination data and medical consultation data of the individual, the information processing device including: patient attribute information acquisition means that acquires information of at least one attribute of a patient who is the individual; a corresponding information database that stores corresponding information representing correspondence relationships between the treatment guideline, including an effect thereof on a predetermined disease symptom, and at least one attribute; corresponding information acquisition means that acquires corresponding information relating to a disease symptom of which the patient is aware from the corresponding information database; optimal treatment guideline calculation means that, on the basis of the patient attribute information acquired by the patient attribute information acquisition means and the corresponding information acquired by the corresponding information acquisition means, calculates for the patient a treatment guideline for the disease symptom of which the patient is aware; effect analysis means that analyzes an effect of the treatment guideline calculated by the optimal treatment guideline calculation means on the patient when the treatment guideline has been applied to the patient; and corresponding information update means that, on the basis of analysis results of the effect analysis means, updates the corresponding information of the treatment guideline, including updating the type of the attributes.

In order to achieve the object described above, an aspect of the information processing device of the present invention includes: a corresponding information database that stores corresponding information representing correspondence relationships between a medicine dosage, including an effect thereof on a predetermined disease symptom, and at least one attribute; patient attribute information acquisition means that acquires information of at least one attribute of the patient; corresponding information acquisition means that acquires corresponding information relating to a disease symptom of which the patient is aware from the corresponding information database; optimal dosage calculation means that, on the basis of the patient attribute information acquired by the patient attribute information acquisition means and the corresponding information acquired by the corresponding information acquisition means, calculates for the patient an optimal medicine dosage for the disease symptom of which the patient is aware; effect analysis means that analyzes an effect of the medicine on the patient when the medicine has been administered to the patient in the dosage calculated by the optimal dosage calculation means; corresponding information update means that, on the basis of analysis results of the effect analysis means, updates the corresponding information of the medicine, the corresponding information including the type of the attribute; and separate effect analysis means that, on the basis of other information other than the patient attribute information, analyzes a separate effect from the effect that is analyzed of the medicine that is analyzed by the effect analysis means.

### Effects of the Invention

According to the present invention, methods may be established for building medical big data taking privacy into consideration, deriving more appropriate medicine dosages in accordance with medicine dosages and attribute information of patients, and discovering, in addition to the effect of a medicine on one disease symptom, effects on other disease symptoms.

### BRIEF DESCRIPTION OF THE DRAWINGS

FIG. 1 is a diagram illustrating structures of an information processing system according to an embodiment of the present invention.

FIG. 2 is a block diagram illustrating hardware structures of a server 2 of the information processing system illustrated in FIG. 1, which server 2 serves as an embodiment of the present invention.

FIG. 3 is a functional block diagram illustrating an example of functional structures for executing optimal dosage setting control among functional structures of a patient terminal 1 and the server 2.

FIG. 4 is a table illustrating a specific example of information other than patient attribute information.

FIG. 5 is a diagram illustrating schematics of a service provided via a medical institution.

FIG. 6 is a graph illustrating an example of an initial dosage set by the service.

FIG. 7 is a diagram illustrating schematics of the service when provided via plural medical institutions.

FIG. 8 is a graph illustrating changes over time in the physical condition of a patient in the related art.

FIG. 9 is a graph comparing respective changes over time in the physical condition of patients when the present system is and is not used.

FIG. 10 is a diagram illustrating an example of an alternative mode of the service.

### PREFERRED MODE FOR CARRYING OUT THE INVENTION

In the following, embodiments of the present invention are explained using the attached drawings.

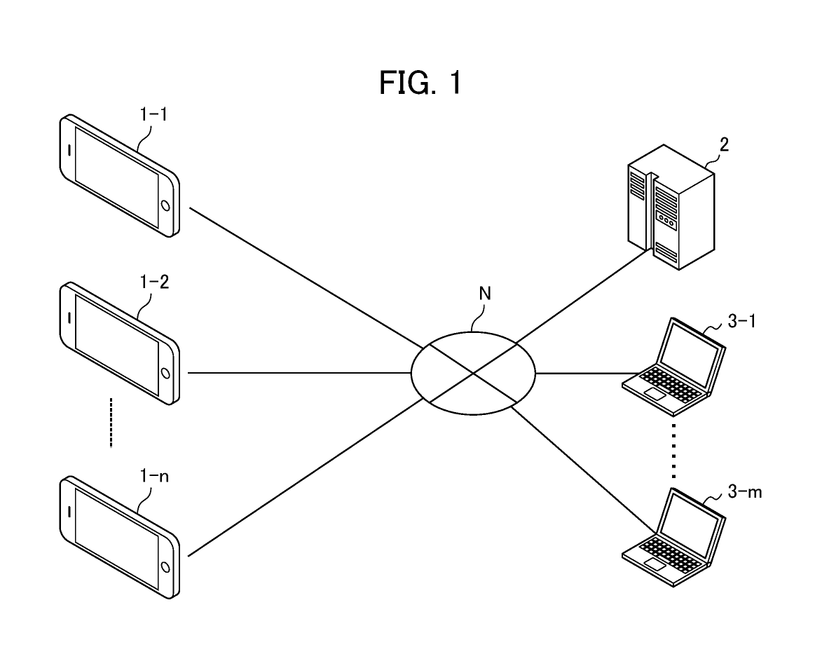

FIG. 1 illustrates the structure of an information processing system according to an embodiment of the present invention. The information processing system illustrated in FIG. 1 is a system that includes n (n is an arbitrary integer that is at least 1) patient terminals 1-1 to 1-n, a server 2, and m (m is an arbitrary integer that is at least 1) medical terminals 3-1 to 3-m. The patient terminals 1-1 to 1-n are used by n respective patients. The medical terminals 3-1 to 3-m are used by m respective medical staff. Each of the patient terminals 1-1 to 1-n, the server 2, and each of the medical terminals 3-1 to 3-m are connected to one another by a network N such as the Internet and the like

The server 2 provides a running environment for setting treatment guidelines such as medicine dosages and the like for each of the patient terminals 1-1 to 1-n, and provides various individual services relating to setting treatment guidelines such as medicine dosages and the like, which are executed at each of the patient terminals 1-1 to 1-n. In the present embodiment, a service that sets a treatment guideline such as an optimal medicine dosage or the like in accordance with the attributes of a patient is applied as one of these services.

Below, where there is no need to individually distinguish the respective patient terminals 1-1 to 1-n, the same are referred to in general as “the patient terminals 1”, and where there is no need to individually distinguish the respective medical terminals 3-1 to 3-m, the same are referred to in general as “the medical terminals 3”.

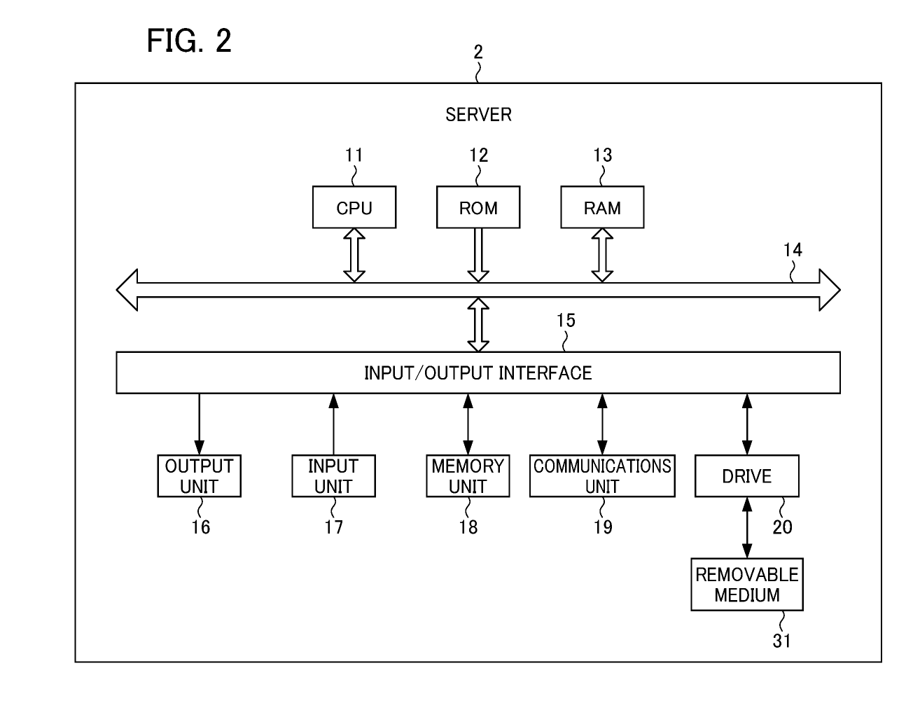

FIG. 2 is a block diagram illustrating hardware structures of the server 2 of the information processing system illustrated in FIG. 1, which server 2 serves as an embodiment of the present invention.

The server 2 is equipped with a central processing unit (CPU) 11, a read-only memory (ROM) 12, a random access memory (RAM) 13, a bus 14, an input/output interface 15, an output unit 16, an input unit 17, a memory unit 18, a communications unit 19 and a drive 20.

The CPU 11 executes various processes in accordance with a program stored in the ROM 12 or a program loaded into the RAM 13 from the memory unit 18. Data and suchlike that is required for the execution of various processes by the CPU 11 is stored in the RAM 13 as appropriate.

The CPU 11, the ROM 12 and the RAM 13 are connected to one another via the bus 14. The input/output interface 15 is also connected to the bus 14. The output unit 16, the input unit 17, the memory unit 18, the communications unit 19 and the drive 20 are connected to the input/output interface 15.

The output unit 16 is structured with a display and a speaker or the like, and outputs images and sound or the like. The input unit 17 is structured with a keyboard and a mouse or the like, and inputs various kinds of information. The memory unit 18 is structured with a hard disc, a dynamic random access memory (DRAM) or the like, and memorizes various kinds of data. The communications unit 19 controls communications with other equipment (in the example in FIG. 1, the patient terminals 1 and the medical terminals 3) via the network N, including the Internet.

The drive 20 is provided as required. A removable medium 31 formed with a magnetic disk, an optical disk, a magneto-optical disk, a semiconductor memory, or the like is mounted in the drive 20 as appropriate. As required, a program read from the removable medium 31 by the drive 20 is installed in the memory unit 18. Similarly to the memory unit 18, the removable medium 31 may memorize the various kinds of data that are memorized in the memory unit 18.

By interoperation of various kinds of hardware and various kinds of software at the server 2 side of FIG. 2, services for building medical big data from the medical terminals 3 and setting optimal medicine dosages at the patient terminals 1 are enabled. That is, the information processing system according to the present embodiment may execute the following control as control (below referred to as optimal dosage setting control) for setting optimal amounts of medicine to be administered to patients on the basis of patient attributes entered through the patient terminals 1.

Many patients take a medicine that is likely to be effective for a disease symptom that the patient is aware of in accordance with a predetermined dosage that is set in advance. However, optimal medicine dosages for patients differ depending on patient attributes (for example, height, weight, gender and age). Accordingly, the server 2 according to the present embodiment collects medical data from the medical terminals 3 and creates big data, sets medicine dosages on the basis of patient attributes, analyzes the effects on patients when the medicine is administered in those dosages and, on the basis of the analysis results, generates or updates optimal dosages. By repeatedly executing this processing sequence for large numbers of patients, the server 2 may determine optimal medicine dosages corresponding to patient attributes. Furthermore, a medicine may both have an effect on one predetermined disease symptom and have effects on plural other disease symptoms. Accordingly, on the basis of attributes, bio-information and so forth of patients to whom a medicine is administered, the server 2 according to the present embodiment may conduct analyses encompassing effects of the medicine on disease symptoms other than a disease symptom of which the patient is aware.

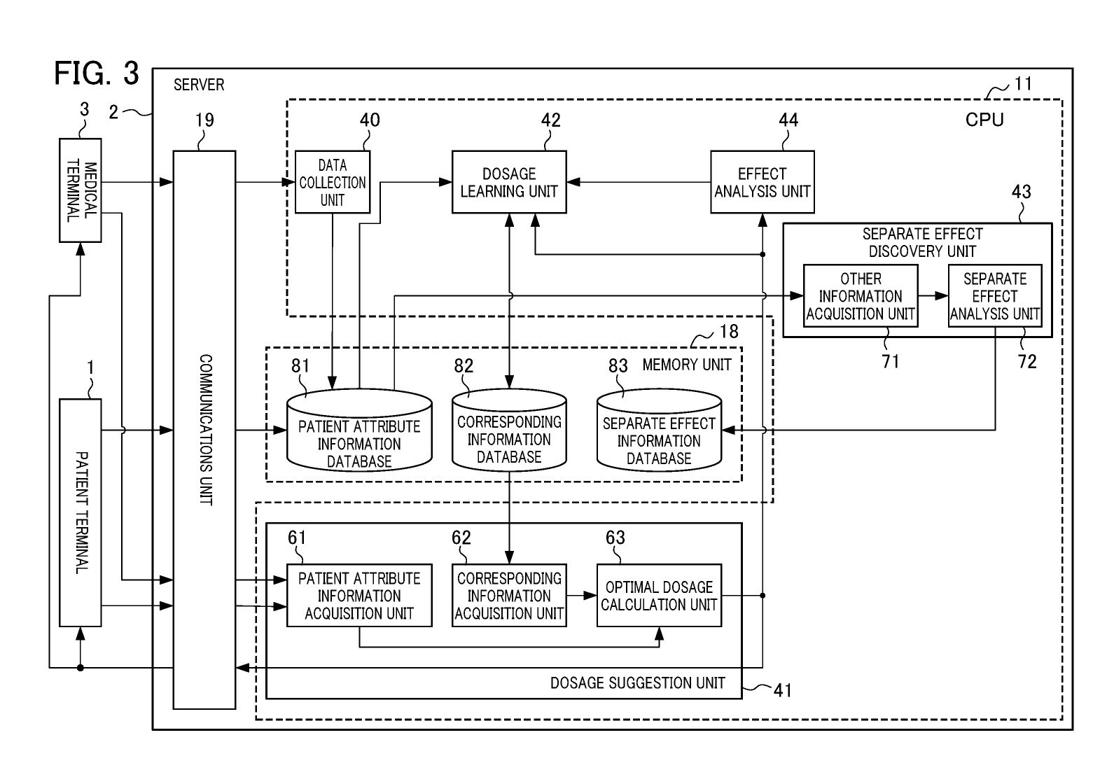

The patient terminals 1, server 2 and medical terminals 3 in FIG. 1 that are to execute the optimal dosage setting control described above have functional structures as illustrated in FIG. 3. FIG. 3 is a functional block diagram illustrating an example of functional structures for executing the optimal dosage setting control among functional structures of the patient terminals 1 and the server 2.

As illustrated in FIG. 3, each patient terminal 1 is a terminal that is operated by a patient and includes at least a function for entering patient attribute information. The patient attribute information referred to here is information representing at least one of height, weight, gender, age and the like of the patient. The patient terminal 1 may send the entered patient attribute information to the server 2 and suggest a medicine dosage, which is suggested by the server 2 as likely to be optimal, to the patient.

As illustrated in FIG. 3, the medical terminal 3 is a terminal that is operated by medical staff and includes at least a function for entering health examination information and identifiers assigned in order to specify individuals in a predetermined group. The identifier referred to here may be the “My Number” ID issued by the government of Japan. The Japanese Ministry of Health, Labor and Welfare will in future require presentation of the My Number ID to medical institutions when accessing medical care, and personal medical information may be collected. Therefore, usage of the My Number system is excellent in regard to promulgating the system according to the present invention. Similarly, the Japanese Ministry of Finance associates My Number IDs with bank accounts, brokerage accounts, and insurance subscriptions. Thus, personal financial information may be collected. Therefore, usage of the My Number system is excellent in regard to promulgating the system according to the present invention. In the context of the My Number system, usage of the My Number system is excellent in regard to widely applying the system according to the present invention to assets that may be utilized for penalties under the law governing the My Number system. The health examination information referred to here includes information representing at least one attribute among the height, weight, gender, age and the like of an examinee receiving a health examination. The medical terminal 3 may send the entered health examination information to the server 2 and suggest a treatment guideline to the medical staff. Treatment guidelines include medicine dosages suggested as likely to be optimal by the server 2 and suchlike.

As illustrated in FIG. 3, the CPU 11 of the server 2 that communicates with the patient terminals 1 and the medical terminals 3 functions as a data collection unit 40, a dosage suggestion unit 41, a dosage learning unit 42, and a separate effect discovery unit 43.

The second identifier capable of identifying a person is generated on the basis of the first identifier that is assigned in order to specify the person in the group. The data collection unit 40 collects health examination data and medical consultation data for the person in association with the second identifier, and stores the data in a patient attribute information database 81, which is a region of the memory unit 18. On the basis of the patient attribute information entered through the patient terminal 1, the dosage suggestion unit 41 suggests a medicine dosage that is optimal for the patient to the patient via the patient terminal 1. The dosage learning unit 42 acquires, from an effect analysis unit 44, information on whether or not the medicine is effective or not and the like from the patient to whom the medicine has been administered in a dosage suggested by the dosage suggestion unit 41. The dosage learning unit 42 uses this information for learning, and generates or updates corresponding information representing correspondence relationships between various attributes and optimal medicine dosages. On the basis of the patient attribute information and other information (for example, bio-information about the blood of the patient and the like), the separate effect discovery unit 43 discovers separate effects of the medicine administered to the patient other than a disease symptom of which the patient is aware. The effect analysis unit 44 analyzes effects on the patient to whom the medicine has been administered in the dosage suggested as optimal by the dosage suggestion unit 41, and provides analysis results to the dosage learning unit 42.

The patient attribute information database 81, a corresponding information database 82 and a separate effect information database 83 are provided at partial regions of the memory unit 18 of the server 2.

The patient attribute information database 81 stores health examination information and consultation information. The patient attribute information database 81 also stores patient attribute information. The health examination information, consultation information and patient attribute information referred to here include, as described above, information capable of specifying at least one attribute of a patient such as, for example, height, weight, gender, age and the like. The corresponding information database 82 stores information representing correspondence relationships between medicine dosages that are effective for disease symptoms and at least one attribute of patients. The separate effect information database 83 stores information about effects on symptoms other than a disease symptom for which a medicine is currently marketed or the like as being effective.

Below, respective functional blocks of the dosage suggestion unit 41, the dosage learning unit 42 and the separate effect discovery unit 43 are described in more detail.

The dosage suggestion unit 41 includes a patient attribute information acquisition unit 61, a corresponding information acquisition unit 62 and an optimal dosage calculation unit 63. The patient attribute information acquisition unit 61 acquires at least one attribute of a patient that is entered through the patient terminal 1. The corresponding information acquisition unit 62 acquires, from the corresponding information database 82, corresponding information representing a correspondence relationship between medicine dosages that are effective for a disease symptom of which a patient is aware and at least one attribute. The optimal dosage calculation unit 63 calculates an optimal medicine dosage for a disease symptom of which a patient is aware on the basis of the patient attribute information and the corresponding information.

The dosage learning unit 42 learns a correspondence relationship between various types of the attributes of patients and optimal medicine dosages, for example, as follows. X1 (weight) and X2 (height) are set as initial parameters of the patient attribute information. The parameters X1 and X2 are entered, and Y=aX1+bX2 is specified as a function f(X1,X2) that outputs a dosage Y. In this equation, a and b are mutually independent coefficients. The dosage learning unit 42 may learn by, for example, the values of the parameters X1 and X2 being appropriately altered and the actual effects of dosages Y outputted by the function f(X1,X2) being entered. Hence, the dosage learning unit 42 may update the coefficients a and b in the function f(X1,X2) to be optimal. The dosage learning unit 42 also derives hypotheses from previous learning results. If it is determined that an optimal dosage cannot be derived with parameter X2, for example, the dosage learning unit 42 discards parameter X2, employs a new parameter X3 (such as gender), and specifies a new function f(X1,X3) that inputs the parameters X1 and X3 and outputs a dosage Y. In this example, the function f(X1,X3) is specified with the output Y=aX1+cX3. At this time, there is a substantial likelihood that the coefficients a and c are not optimal. Accordingly, the dosage learning unit 42 may learn by the values of the parameters X1 and X3 being appropriately altered and the actual effects of dosages Y outputted from the function f(X1,X3) being entered, and hence the dosage learning unit 42 may update the coefficients a and c in the function f(X1,X3) to be optimal. Further, the dosage learning unit 42 may increase the number of parameters to three parameters, X1 to X3, and specify a new function f(X1,X2,X3) that inputs the parameters X1 to X3 and outputs the dosage Y. In this example, the function f(X1,X2,X3) is specified with the output Y=aX1+bX2+cX3. At this time, there is a substantial likelihood that the coefficients a, b and c are not optimal. Accordingly, the dosage learning unit 42 may learn by the values of the parameters X1 to X3 being appropriately altered and the actual effects of dosages Y outputted from the function f(X1,X2,X3) being entered, and hence the dosage learning unit 42 may update the coefficients a, b and c in the function f(X1,X2,X3) to be optimal.

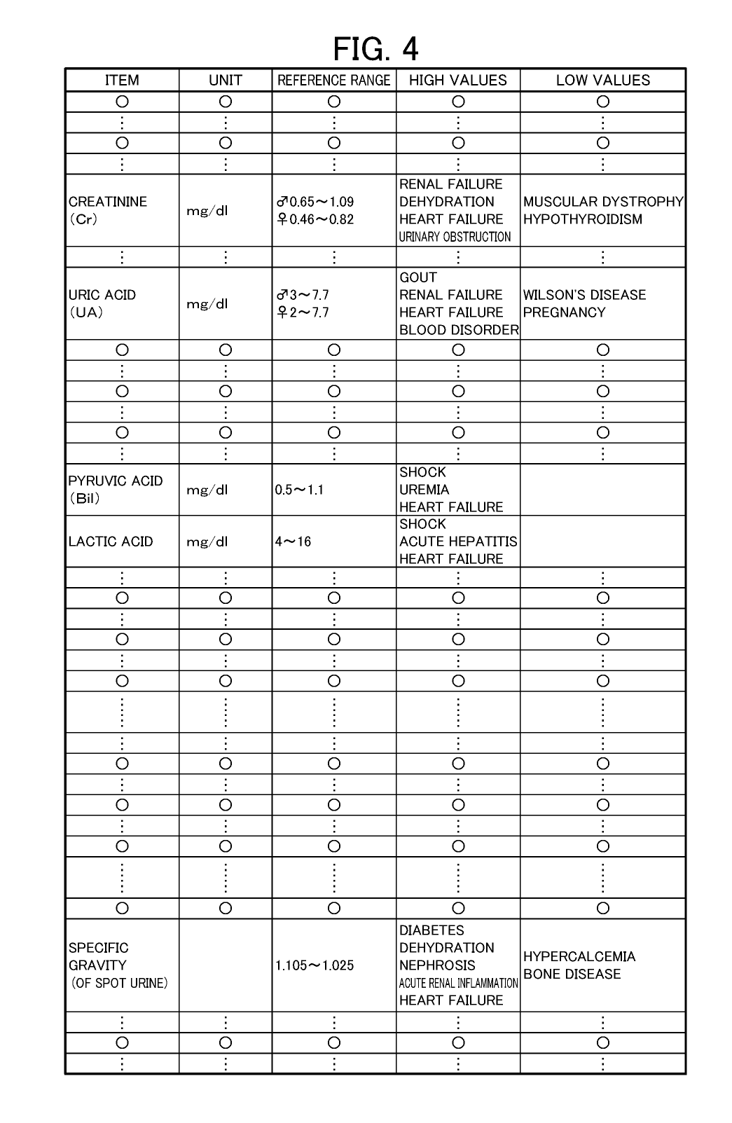

The separate effect discovery unit 43 includes an other information acquisition unit 71 and a separate effect analysis unit 72. The other information acquisition unit 71 acquires other information other than the patient attribute information from the patient attribute information database 81. On the basis of the other information acquired by the other information acquisition unit 71, the separate effect analysis unit 72 analyzes effects of the medicine analyzed by the effect analysis unit 44 on disease symptoms other than the disease symptom that is analyzed by the effect analysis unit 44. A specific example of other information other than patient attribute information is depicted in FIG. 4.

FIG. 4 is a table illustrating a specific example of other information other than patient attribute information. FIG. 4 is organized into item, unit, reference range, high values and low values. The information other than patient attribute information is based on results of various tests such as a blood biochemistry test, a hematology test, a serology test, a urine analysis, a kidney function test, an endocrine function test, a circulatory function test and the like. For example, as illustrated in FIG. 4 for creatinine (Cr), values higher than the reference range may indicate renal failure, dehydration, heart failure or urinary obstruction, and values lower than the reference range may indicate muscular dystrophy or hypothyroidism. Further, as illustrated in FIG. 4 for uric acid (UA), values higher than the reference range may indicate gout, renal failure, heart failure or a blood disorder, and values lower than the reference range may indicate Wilson's disease or pregnancy. As illustrated in FIG. 4 for pyruvic acid, values higher than the reference range may indicate shock, acute hepatitis or heart failure. As illustrated in FIG. 4 for lactic acid, values higher than the reference range may indicate shock, uremia or heart failure. As illustrated in FIG. 4 for specific gravity (of spot urine), values higher than the reference range may indicate diabetes, dehydration, nephrosis, acute renal inflammation or heart failure, and values lower than the reference range may indicate hypercalcemia or bone disease. For example, if a patient is aware of a disease symptom of heart failure, a medicine for heart failure has been administered, and the value of creatinine (Cr) found in a blood biochemistry test falls from a high value to the reference range, the separate effect analysis unit 72 may discover that the medicine for heart failure also has effects on symptoms of renal failure, dehydration and urinary obstruction. As another example, if a patient is aware of a disease symptom of gout, a medicine for gout has been administered, and the value of uric acid (UA) found in a blood biochemistry test falls from a high value to the reference range, the separate effect analysis unit 72 may discover that the medicine for gout also has effects on symptoms of renal failure, heart failure and blood disorders. As another example, if a patient is aware of a disease symptom of uremia, a medicine for uremia has been administered, and the value of pyruvic acid found in a hematology test falls from a high value to the reference range, the separate effect analysis unit 72 may discover that the medicine for uremia also has effects on symptoms of shock and heart failure. As another example, if a patient is aware of a disease symptom of acute hepatitis, a medicine for acute hepatitis has been administered, and the value of lactic acid found in a hematology test falls from a high value to the reference range, the separate effect analysis unit 72 may discover that the medicine for acute hepatitis also has effects on symptoms of shock and heart failure. As still another example, if a patient is aware of a disease symptom of diabetes, a medicine for diabetes has been administered, and the value of specific gravity (of spot urine) found in a urine analysis falls from a high value to the reference range, the separate effect analysis unit 72 may discover that the medicine for diabetes also has effects on symptoms of dehydration, nephrosis, acute renal inflammation and heart failure.

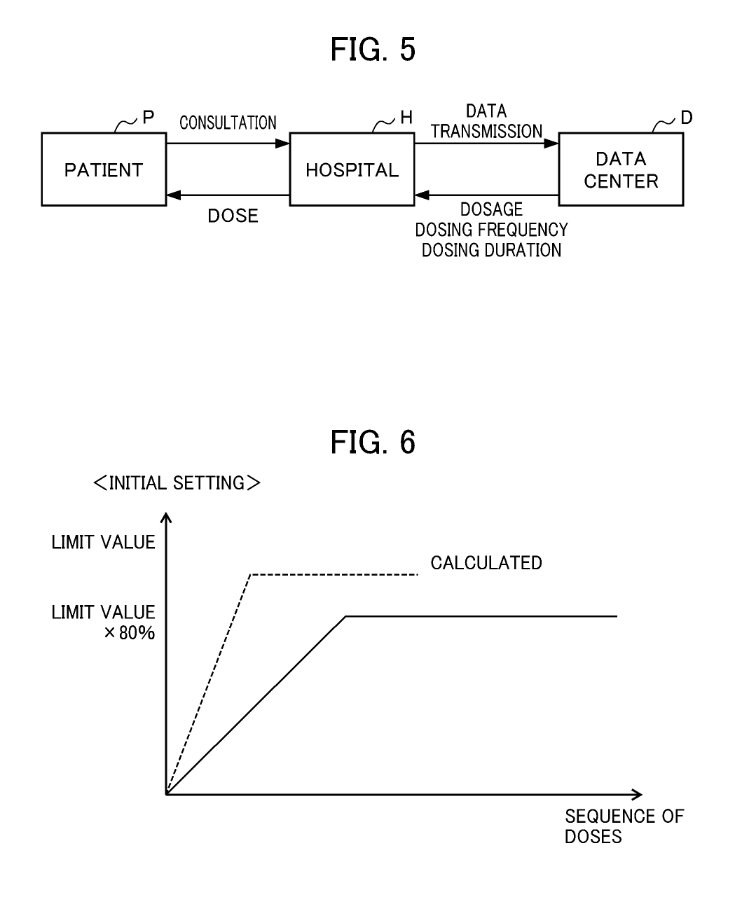

Above, a mode in which a patient themself operates the patient terminal 1 and receives a service is described. Next, a mode in which a patient receives a service via a medical institution is described. FIG. 5 is a diagram illustrating schematics of the service provided via the medical institution. This service is provided in a system constituted by a patient P, a hospital H and a data center D. The patient P visits the hospital H and receives a consultation or a health examination from a doctor. The hospital H provides medical services to the patient P through at least one doctor. The doctor sends data from the consultation or health examination to the data center D. The data center D is managed by a service provider and provides a service to the hospital H and doctor to set a dosage, number of doses and dosing duration of a medicine.

As a specific example of a medical service provided to a patient P, administration of a cell culture supernatant fluid as an intravenous drip or nasal drip can be considered. Fluid components (growth factors, cytokines, lipids, nucleic acids and the like) that are secreted in a supernatant produced when stem cells are cultured are administered into the body, with the result that endogenous stem cells are activated and the stem cells are induced to heal a damaged area. However, if a dosage exceeds a suitable amount, there is a risk of cytokine release syndrome occurring. Diseases that may be targeted include stroke, dermatitis, spinal damage, lung disease, liver disease, diabetes and so forth. The range of treatment is likely to expand to other diseases with future research. Big data of patient dosages of cell culture supernatant fluids can be created by the present invention. Thus, the prevention of cytokine release syndrome may be reliably assured and applications of cell culture supernatant fluids may expand and advance.

FIG. 6 is a diagram illustrating an example of an initial dosage set by the present service. In the present embodiment, the data center D finds a dosage limit value by a predetermined calculation and sets a maximum dosage by multiplying the limit value by a safety factor. The limit value is a safe maximum dosage calculated from patient attributes. The safety factor for the maximum dosage is a numerical value between 0 and 1. For example, 0.8 may be employed. Similarly, a dosage duration is found by a calculation of a limit value of a dosage increase rate, and the dosage increase rate is set by multiplying this limit value by a safety factor. The safety factor of the dosage increase rate is a numerical value between 0 and 1. For example, 0.5 may be employed.

As patient data that is used in the calculation of the limit values, for example, age, gender, weight, height, body temperature, blood pressure, pulse rate, blood type, total body fluid, urine, and external injury image data may be used. The calculations may reduce a number of doses to a suitable value, for example, by clinical test values from various cases being referred to, therapeutic effects being measured, and dosages being repeatedly adjusted. Use of the present invention may be spread through, for example, a business model that charges information provision fees.

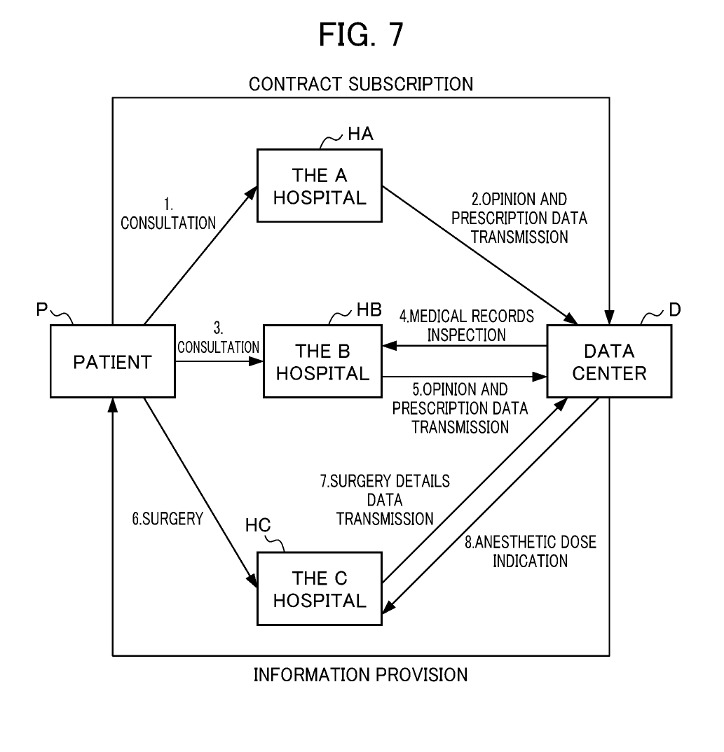

FIG. 7 is a diagram illustrating schematics of the service when provided via plural medical institutions. In FIG. 7, the hospital H in FIG. 5 is replaced with three hospitals, “the A hospital” HA, “the B hospital” HB and “the C hospital” HC. A patient P uses the same personal identifier (for example, the My Number ID) at all three of the A hospital HA, the B hospital HB and the C hospital HC. Accordingly, the data center D regards the patient P as the same person and, even though patient P is visiting different hospitals, patient P may receive treatments such as consultations, surgery and the like from different doctors on the basis of the same data. Input data may include, for example, personal data (including genetic information), prescription records, clinical records, surgical records, family disease history, treatment status of diseases currently being treated, data from periodic check-ups, daily data collected by wearable devices and portable devices, data shared from home medical devices, details of food and drink consumption, sleeping times and so forth. Output data may include, for example, anesthetic doses for surgery, anti-cancer drug dosages, painkiller dosages for palliative care, doses of prescribed medicines, proposals for pre-emptive and precautionary treatments, proposals for mixed treatments, proposals for health management and exercise programs, proposals for dietary management, dietary restrictions and recommended menus, disease prognoses, selections and alternatives of recommended hospitals, recommendations and alternatives of health foods and supplements, and so forth. Accordingly, details of treatments at other hospitals may be checked during a consultation, efficient use and combinations of medicines when a patient is attending plural medical institutions may be adjusted, and medical errors may be discovered and prevented. As a specific example of a case that might be prevented according to the present invention, an incident in which lung cancer signs were missed, which occurred at Jikei University Hospital, Tokyo in 2016, can be mentioned. A doctor in the radiology department wrote in an imaging report that primary lung cancer was apparent and should be followed up promptly. However, the attending doctor and the doctor with primary responsibility for out-patients did not check the report, the signs of lung cancer were missed for over a year, and the cancer progressed to a state that could not be treated by surgery or anti-cancer drugs. This is an example of a case that could have been avoided if medical data for the one patient could have been inspected by multiple medical staff. Use of the present invention may be spread by, for example, a business model in which a patient contractually subscribes to a data center, at the end of each consultation the patient instructs the hospital to send data to the data center, a transmission fee is paid from the data center to the hospital, the patient is billed with information provision fees when medical records are inspected or medication is instructed, and commissions are earned by various agents.

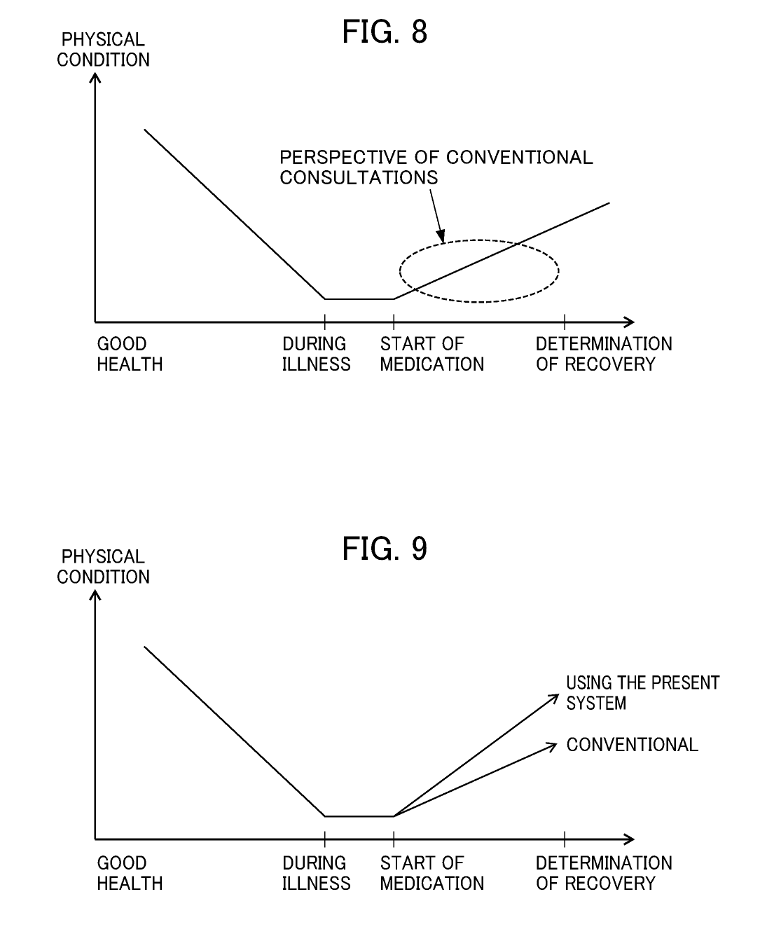

FIG. 8 is a graph illustrating changes over time in the physical condition of a patient in the related art. The vertical axis represents the physical condition of the patient, with higher positions representing the patient being in good health. In the present invention, various clinical test values may be collected to serve as base values in a period in which the health condition of the patient is good. Accordingly, clinical test values at the onset of a disease and after the start of medication may be compared with the base values and treated as a recovery rate.

In the present invention, personal differences may be taken into account by analyzing base value data from birth. For example, if a person whose average body temperature is 36° C. and a person whose average body temperature is 37° C. both have the same body temperature of 38° C. at the onset of illness, their conditions may be understood as being different.

In the present invention, weightings of the parameters that are referred to may be altered between a case of administering medicine over a plural number of occasions and a case of administering medicine on only one occasion. For example, personal data may be prioritized as the number of doses increases in a case of continuous medication, and big data may be prioritized in a case of medication on a single occasion and for initial doses in a case of continuous medication.

The present invention may be applied to, for example, a case of treating diabetes by dosing with a cell culture supernatant fluid. In this case, an occurrence of cytokine release syndrome caused by excessive dosing of the cell culture supernatant fluid would be a problem. For this case in the present invention, the dosage for an initial dose may be set on the basis of big data. Similarly in the present invention, dosages for second and subsequent doses may be increased or reduced in accordance with clinical test values from the patient (for example, urine pH, urine sugar and urine ketone bodies from a urine analysis, and a blood sugar value and hemoglobin value from a hematology test).

FIG. 9 is a graph comparing respective changes over time in the physical condition of patients when the present system is and is not used. The vertical axis represents the physical condition of the patients, with higher positions representing the patients being in good health. According to the present invention, the following effects may be expected. Firstly, improved therapeutic results may be expected due to appropriate dosing of a drug. Because of both consistent administration of medicine based on previous cases and the setting of dosages according to individual conditions, more effective treatment is enabled. Consequently, as illustrated in FIG. 9, the rate of recovery in the physical condition of a patient may be improved.

Beside the effect described above, the following effects, which are not illustrated in the drawings, are provided by the present invention. Secondly, unsuitable doses of drugs may be avoided. For example, a propofol dosing accident that occurred at Tokyo Women's Medical University Hospital in February 2016 can be mentioned as an example of a specific incident that might be prevented according to the present invention. The use of propofol as a sedative during artificial respiration in infant intensive care is contraindicated but a large dose was administered without the consent of the family, as a result of which a boy aged two years and ten months died. This accident might have been avoided if dosages were set using big data.

Thirdly, according to the present invention, unused medicines may be managed, limited and kept from resale. See the attached document. When a system identifies excessive prescription, the system stops the prescription.

Fourthly, pre-emptive treatments may be disseminated and promoted. According to the present invention, rather than values of body weight, body temperature and other clinical test values being determined only during examinations at the onset of a disease, changes from the past may be acquired. Therefore, the progress of disease onset and variations in the disease may be inferred and treatment may be started promptly.

Fifthly, payment of medical fees may be simplified. If medical fee payments are transferred from the accounts of all patients, hospital front desk work may be simplified, which will help to reduce crowding in hospitals.

Sixthly, fraudulent billing may be prevented. If treatment information and payment information are associated according to the present invention, fraudulent billing for health insurance payments resulting from dishonest behavior in hospitals may be prevented.

Seventhly, the emergence of multilateral monitoring functions based on the sharing of medical information may be expected. According to the present invention, the details of a consultation in a medical institution may be checked by other doctors and by an AI, which will contribute to discovering second opinion shopping, misdiagnoses and medical malpractice.

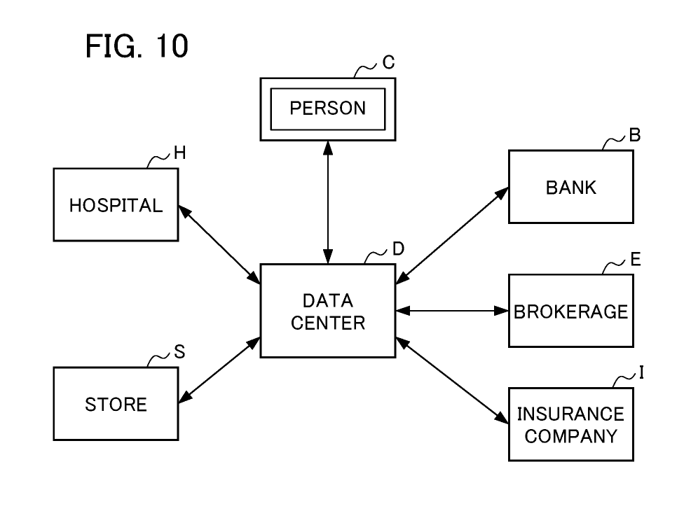

FIG. 10 is a diagram illustrating an example of an alternative mode of the service. A My Number ID is used as follows for the management of personal health and finances. As a first usage example, use of a My Number ID for a medical consultation is described. A person C presents their My Number ID to a hospital H when receiving a consultation as a patient. Hospital H sends the details of the consultation to a data center D in association with the My Number ID of person C. As a payment for the consultation to hospital H, person C has their fingerprint authenticated and, using a personal financial ID based on their My Number ID, instructs a bank B to transfer the money. The details of the consultation at hospital H are administered at data center D with the My Number ID of person C, are analyzed by an AI, and a subsequent therapeutic guideline, disease prognosis and medicine prescription are determined.

Now, an example of a process in which a person C who is a diabetes patient receives a dose of a cell culture supernatant fluid at a hospital H is described in specific terms. First, person C presents a personal ID card at the front desk of hospital H and their My Number ID is acquired. Person C proceeds to a consultation department, a doctor in hospital H requests personal data for person C from a data center D, and a summary of their medical history and recent medication is displayed on a medical terminal. The doctor in hospital H operates the medical terminal and clicks on buttons displayed on a screen in the order “Dosage for today” and “Cell Culture Supernatant fluid”. In response, clinical test values at a time of good health, a time of disease onset, and after medication are compared, and a dosage is calculated. The calculated dosage is increased or reduced in accordance with the results of the comparison of values. When the doctor in hospital H operates the medical terminal and sends data to data center D, consultation details and prescriptions from other hospitals may be checked. Therefore, multiple medication and duplicate medication may be managed. Hence, the doctor in hospital H may administer the cell culture supernatant fluid to Person C. Person C then proceeds to a payment department and hospital H presents a treatment details statement to person C. This treatment details statement includes a charge for data provision services from data center D. Person C has their fingerprint authenticated at the front desk of hospital H and a treatment fee is transferred from person C's account at a bank B to hospital H. At this time, hospital H may ask person C to present their ID card at the front desk again.

As a second usage example, use of a My Number ID during self-directed health management is described. A person C sends image data showing their meals to a data center D in association with their My Number ID. At data center D, calories are calculated and so forth, and various kinds of data are administered in association with person C's My Number ID. Periodically, the various kinds of data accumulated in association with person C's My Number ID are analyzed by an AI, and menu recommendations and restrictions are sent to person C.

As a third usage example, use of a My Number ID during payment in a store is described. When shopping in a store S, a person C presents a personal financial ID based on their My Number ID and has their fingerprint authenticated. Details of the shopping based on the personal financial ID based on the My Number ID are judged by an AI, and a transfer instruction is sent to a bank B. The details judged by the AI identify the person according to their interests, preferences, location, prices and the like.

As a fourth usage example, use of a My Number ID in a financial institution or the like is described. A balance of funds held by a bank B, brokerage E or insurance company I is sent to a data center D in association with a personal financial ID based on the My Number ID of a person C. At the end of the year, data for a tax declaration for that year is created from balance data and sent to person C. A portfolio analysis is conducted by an AI. The health condition of person C is analyzed by the AI in accordance with the personal financial ID based on the My Number ID, and an appropriate level of insurance is selected and sent to person C. A preliminary calculation of inheritance tax is conducted on the basis of the personal financial ID based on the My Number ID.

As a fifth usage example, use of a My Number ID when purchasing an over-the-counter medicine—for judging suitability, setting a usage method and a usage amount, and paying the price—is described. A store S displays details of components and the like as barcodes on the packages of over-the-counter medicines. A person C visiting store S scans a barcode with a smartphone and the barcode is sent to a data center D. At data center D, the personal data of person C is analyzed by an AI, and the suitability, usage method and usage amount are sent back. When person C is paying the purchase price, their My Number ID and fingerprint are authenticated, a fund transfer instruction is sent from data center D to a designated account, and the price is transferred to store S. The data center D saves the data in a purchase history. Person C subsequently sends dosages. Data center D monitors duplicative purchases, previous similar medicines and unused medicines, and sends warnings to person C. Data center D collates annual information and sends data for a tax deduction for medical expenses to person C. Data center D suggests appropriate treatments and treatment locations according to an AI to person C. Data center D recommends suitable food menus and rest schedules according to the AI to person C. A link from a food menu may suggest a restaurant booking site. A link from a rest schedule may suggest a travel booking site. Data center D calculates a life expectancy for person C according to the AI, and selects and suggests suitable life insurance. Data center D calculates an estimate of inheritance tax for person C according to the AI, and selects and suggests a suitable asset portfolio.

An embodiment of the present invention is described above but it should be noted that the present invention is not limited to the above embodiment; any modifications and improvements thereto within a scope in which the object of the present invention may be achieved are to be encompassed by the present invention.

For example, the functional structures in FIG. 3 are merely examples and are not particularly limiting. That is, it is sufficient if a function capable of executing the whole of an above-described sequence of processing is provided at the information processing system; the kinds of functional blocks to be used for executing this function are not particularly limited by the example in FIG. 3. Moreover, the locations of functional blocks are not particularly limited by FIG. 3 and may be arbitrary. For example, functional blocks of the server 2 may be transferred to the patient terminals 1 or the like. Conversely, functional blocks of the patient terminal 1 that are not shown in FIG. 3 may be transferred to the server 2 or the like. A single functional block may be configured by a single unit of hardware, a single unit of software, or any combination thereof.

In a case in which the processing of the functional blocks is to be executed by software, a program configuring the software is installed from a network or a storage medium into a computer or the like. The computer may be a computer embedded in dedicated hardware. Alternatively, the computer may be a computer capable of executing various functions by installing various programs. For example, as an alternative to a server, the computer may be a smartphone, a personal computer or the like.

As well as a removable medium that is distributed separately from the main body of the equipment for supplying the program, a recording medium containing such a program may be constituted by a recording medium or the like that is supplied in a state of being incorporated in the main body of the equipment.

It should be noted that the steps in the present specification describing each program recorded in the storage medium include not only processing executed in a time series following this sequence, but also processing that is not necessarily executed in a time series but is executed in parallel or individually. Moreover, the term “system” as used in the present specification is intended to include the whole of equipment constituted by plural devices, plural units and the like.

In other words, an information processing device in which the present invention is employed may be embodied in various modes including the configuration described below. That is, an information processing device in which the present invention is employed includes data collection means (for example, the data collection unit 40 in FIG. 3) that collects at least one of health examination data and medical consultation data relating to an individual in association with a second identifier that is capable of specifying the individual, the second identifier being generated on the basis of a first identifier (for example, a My Number ID) that is assigned in order to specify the individual within a predetermined group (the population of Japan).

Further, an information processing device in which the present invention is employed includes: an information processing device for suggesting a treatment guideline for an individual on the basis of at least one of health examination data and medical consultation data of the individual, the information processing device including: patient attribute information acquisition means (for example, the patient attribute information acquisition unit 61 in FIG. 3) that acquires information of at least one attribute of a patient who is the individual; a corresponding information database (for example, the corresponding information database 82 in FIG. 3) that stores corresponding information representing correspondence relationships between the treatment guideline, including an effect thereof on a predetermined disease symptom, and at least one attribute; corresponding information acquisition means (for example, the corresponding information acquisition unit 62 in FIG. 3) that acquires corresponding information relating to a disease symptom of which the patient is aware from the corresponding information database; optimal treatment guideline calculation means (for example, the optimal dosage calculation unit in FIG. 3) that, on the basis of the patient attribute information acquired by the patient attribute information acquisition means and the corresponding information acquired by the corresponding information acquisition means, calculates for the patient a treatment guideline for the disease symptom of which the patient is aware; effect analysis means (for example, the effect analysis unit 44 in FIG. 3) that analyzes an effect of the treatment guideline calculated by the optimal treatment guideline calculation means on the patient when the treatment guideline has been applied to the patient; and corresponding information update means (for example, the dosage learning unit 42 in FIG. 3) that, on the basis of analysis results of the effect analysis means, updates the corresponding information of the treatment guideline, including updating the type of the attributes. Further, an information processing device in which the present invention is employed includes: patient attribute information acquisition means (for example, the patient attribute information acquisition unit 61 in FIG. 3) that acquires information of at least one attribute of a patient; a patient attribute information database (for example, the patient attribute information database 81 in FIG. 3) that stores the patient attribute information; a corresponding information database (for example, the corresponding information database 82 in FIG. 3) that stores corresponding information representing correspondence relationships between a medicine dosage, including an effect thereof on a disease symptom of which the patient is aware, and at least one attribute; corresponding information acquisition means (for example, the corresponding information acquisition unit 62 in FIG. 3) that acquires the corresponding information; optimal dosage calculation means (for example, the optimal dosage calculation unit 63 in FIG. 3) that, on the basis of the acquired patient attribute information and corresponding information, calculates for the patient an optimal medicine dosage for the disease symptom of which the patient is aware; effect analysis means (for example, the effect analysis unit 44 in FIG. 3) that analyzes an effect of the calculated optimal medicine dosage; corresponding information update means (for example, the dosage learning unit 42 in FIG. 3) that, on the basis of the analysis results, updates the corresponding information, including updating the type of the attributes; separate effect analysis means (for example, the separate effect analysis unit 72 in FIG. 3) that, on the basis of other information other than the patient attribute information, analyzes a separate effect from the analyzed effect on a symptom other than the disease symptom of which the patient is aware; a separate effect information database (for example, the separate effect information database 83 in FIG. 3) that stores information of the separate effect; and other information acquisition means (for example, the other information acquisition unit 71 in FIG. 3) that acquires other information other than the patient attribute information. The meaning of the term “patient” as used herein is broadly defined to include, as well as humans as described in the above embodiment, other subjects of medicine dosing such as, for example, animals and plants. The information processing device that is provided with the data collection means (for example, the data collection unit 40 in FIG. 3) and the information processing device that is provided with the optimal treatment guideline calculation means (for example, the optimal dosage calculation unit in FIG. 3) may be combined in a single information processing device.

Thus, methods are established for building medical big data taking privacy into consideration, deriving more appropriate medicine dosages in accordance with medicine dosages and attribute information of patients, and discovering, in addition to the effect of a medicine on one disease symptom, effects on other disease symptoms. That is, an optimal medicine dosage based on information corresponding to a patient attribute may be set by: collecting health examination data or medical consultation data relating to an individual in association with a second identifier that is capable of specifying the individual, the second identifier being generated on the basis of a first identifier that is assigned in order to specify the individual within a predetermined group; using the collected data to set a medicine dosage on the basis of a patient attribute; analyzing an effect of the dosed medicine; and updating the optimal dosage on the basis of analysis results. Furthermore, analysis of a medicine, including an effect on a disease symptom other than a disease symptom of which a patient is aware, may be conducted on the basis of other information other than the patient attribute information.

### EXPLANATION OF REFERENCE NUMERALS

1 patient terminal, 2 server, 3 medical terminal, 11 CPU, 18 memory unit, 40 data collection unit, 41 dosage suggestion unit, 42 dosage learning unit, 43 separate effect discovery unit, 44 effect analysis unit, 71 other information acquisition unit, 72 separate effect analysis unit, 81 patient attribute information database, 82 corresponding information database, 83 separate effect information database

1. An information processing device comprising data collection means that collects at least one of health examination data and medical consultation data relating to an individual in association with a second identifier that is capable of specifying the individual, the second identifier being generated on the basis of a first identifier that is assigned in order to specify the individual within a predetermined group.

2. An information processing device for suggesting a treatment guideline for an individual on the basis of at least one of health examination data and medical consultation data of the individual, the information processing device comprising:
patient attribute information acquisition means that acquires information of at least one attribute of a patient who is the individual;
a corresponding information database that stores corresponding information representing correspondence relationships between the treatment guideline, including an effect thereof on a predetermined disease symptom, and at least one attribute;
corresponding information acquisition means that acquires corresponding information relating to a disease symptom of which the patient is aware from the corresponding information database;
optimal treatment guideline calculation means that, on the basis of the patient attribute information acquired by the patient attribute information acquisition means and the corresponding information acquired by the corresponding information acquisition means, calculates for the patient a treatment guideline for the disease symptom of which the patient is aware;
effect analysis means that analyzes an effect of the treatment guideline calculated by the optimal treatment guideline calculation means on the patient when the treatment guideline has been applied to the patient; and
corresponding information update means that, on the basis of analysis results of the effect analysis means, updates the corresponding information of the treatment guideline, including updating the type of the attributes.

3. The information processing device according to claim 2, wherein the treatment guideline in the corresponding information database is a medicine dosage,
the treatment guideline calculated by the optimal treatment guideline calculation means is about the medicine dosage,
the effect of the treatment guideline on the patient that is analyzed by the effect analysis means is an effect of the medicine on the patient when the medicine dosage has been administered to the patient, and
the corresponding information of the treatment guideline that is updated by the corresponding information update means includes corresponding information of the medicine.

4. The information processing device according to claim 2, further comprising separate effect analysis means that, on the basis of other information other than the patient attribute information, analyzes a separate effect of the treatment guideline or medicine analyzed by the effect analysis means other than the effect that is analyzed by the effect analysis means.
